(19) United States
(12) Patent Application Publication
(10) Pub. No.: US 2011/0110953 A1
(43) Pub. Date: May 12, 2011

(54) Compound and method for treatment of chronic transplant rejection

(76) Inventor: Dennis Keith Bishop, Ann Arbor, MI (US)

(21) Appl. No.: 12/803,117

(22) Filed: Jun. 17, 2010

Related U.S. Application Data
(60) Provisional application No. 61/269,881, filed on Jun. 29, 2009.

(51) Int. Cl. A61K 39/395; A61P 37/06

(52) U.S. Cl. 424/145.1

(57) ABSTRACT

Methods of treating chronic allograft rejection are provided in the present invention. The method uses anti-CTGF agents, particularly anti-CTGF antibodies, to reduce or reverse the occurrence of fibrosis in the allograft providing prolonged survival of the transplanted organ or tissue.

### STATEMENT REGARDING FEDERALLY SPONSORED RESEARCH

This work was funded in part by Grant Nos. R01 HL070613 and R01 A1061469 from the National Institutes of Health. Accordingly, the Government has certain rights in this invention.

### BACKGROUND

In 2005, over 50,000 solid organ transplants were conducted in the US, Japan and five major European markets: 62% of transplanted organs were kidneys (29,910), followed by livers (23%; 11,333) and cardiothoracic (12%; 5,817). In the US alone, the number of transplantation procedures by organ in 2005 was: Kidney 16,895; Liver, 5,985; Heart, 2,059; and Lung, 1,402 (Transplant Data 1997-2006. Annual Report of the U. S. Organ Procurement and Transplantation Network and the Scientific Registry of Transplant Recipients (2007)). The total number of transplant procedures is expected to increase to 67,112 by 2015. The number of patients living with functional grafts at year end 2005 was approximately 163,631 in the United States alone, indicating a significant patient population. While remarkable progress has been made in the ability to transplant various organs, long term preservation (i.e. greater than one year) of organ function and patient survival suffers primarily because of chronic rejection. For example, one-year survival of transplanted kidneys has steadily improved to where it is now over 90%, but the 10-year survival remains fixed at about 38% (Transplantation. US Renal Data System (http://www.usrds.org/adr.htm). (2007)). Chronic rejection stems from multiple types of insults ranging from ischemia-reperfusion injury during the transplant procedure (Braunwald, E. Heart Disease: A Textbook of Cardiovascular Medicine. W.B. Saunders, New York (2001), recurrent disease, and bouts of acute rejection, and to certain drugs used to combat acute rejection of allografts (tissue obtained from a genetically non-identical member of the same species). The precise manifestations of chronic rejection will vary according to the transplanted organ, but all will exhibit proliferation of myofibroblasts, or related cells, ultimately resulting in fibrosis that leads to loss of function (Mannon, R. B. Therapeutic targets in the treatment of allograft fibrosis. Am J Transplant 6, 867-875 (2006)).

At present no drugs are available for treatment of the fibroproliferative lesions of progressive chronic allograft rejection. Currently transplant patients are placed on a standard triple therapy that includes corticosteroids (e.g., prednisone), antimetabolites (e.g., azathioprine), and calcineurin inhibitors (e.g., tacrolimus, cyclosporine). Mycophenolate mofetil is now being substituted for azathioprine at some centers due to milder bone marrow suppression. Sirolimus (rapamycin) is being used as an inhibitor of T-cell signaling pathways to prevent the response to IL-2 and other cytokines. Sirolimus can be used in conjunction with cyclosporine or tacrolimus, or with mycophenolate mofetil to avoid calcineurin inhibitors. In some cases induction or maintenance of immunosuppression with monoclonal antibodies (OKT3, daclizumab, basiliximab) is appropriate (Hunt, S. pp. 1455-1457, Carpenter et al. pp. 1776-1781, Dienstag, J. L. and Chung, R. T. pp. 1983-1990. In: Harrison's Principles of Internal Medicine, 17th edition. Fauci, A. S. et al. (eds.), (McGraw-Hill, New York, 2008). While treatment with the current therapeutic regimen has reduced the acute allograft rejection, chronic allograft rejection (CR) is a significant barrier to long term graft acceptance.

Manifestations of CR include interstitial fibrosis, occlusion of luminal structures, and progressive loss of graft function (Orosz C G and Pelletier R P. Curr Opin Immunol 1997 9(5):676-680; Paul L C. Transplant Proc 1999 31(4):1793-1795; Waaga A M et al. Curr Opin Immunol 2000 12(5):517-521; Womer K L et al. Semin Nephrol 2000 20(2):126-147; Weiss M J et al. Front Biosci 2008; 13:2980-2988; Mehra M R. Am J Transplant 2006 6(6):1248-1256; Valantine H. J Heart Lung Transplant 2004 23(5 Suppl):S187-193.). As effective therapeutics for chronic rejection are lacking, re-transplantation is currently the only treatment for this condition. New therapeutics for the treatment, prevention or amelioration of chronic allograft rejection are therefore desirable.

### SUMMARY OF THE INVENTION

In one aspect, the present invention provides a method of treating chronic allograft rejection in a subject, wherein the method comprises administering an anti-CTGF agent to the subject. The allograft may be any transplanted tissue or organ that is subject to chronic allograft rejection, including heart, heart valves, liver, lung, kidney, intestines, skin, eye, cornea, pancreas, ligament, tendon, and bone, composite tissue grafts (e.g., hand transplants, face transplants) and multiple organ transplants (e.g., heart-lung transplants, kidney-pancreas transplants). In a preferred embodiment, the allograft is a cardiac (i.e., heart) allograft.

The subject is an individual, preferably a mammal, more preferably a human, who has received or is about to receive an allograft; alternatively, the subject may be a transplanted organ or tissue.

In another aspect, the present invention provides a method of reducing or preventing fibrosis in an allograft. In a further aspect the invention provides a method for prolonging the survival of an allograft in a recipient. In yet another aspect the present invention provides a method of reducing transplant-associated hypertrophy in an allograft. In a particular embodiment the invention provides a method of reducing cardiac hypertrophy associated with cardiac transplantation.

These and other methods of the invention are accomplished by administering an anti-CTGF agent to the recipient of the transplanted organ/tissue and/or to the transplanted organ/tissue. In particular embodiments the anti-CTGF agent is an anti-CTGF antibody, a polynucleotide inhibitor of CTGF expression (for example, an antisense oligonucleotide, siRNA, shRNA, miRNA, or ribozyme) or a small molecule inhibitor of CTGF activity. In a preferred embodiment, the anti-CTGF agent is an anti-CTGF antibody. A preferred anti-CTGF antibody is CLN1 or mAb1, or the antibody produced by ATCC Accession No. PTA-6006, as described in U.S. Pat. No. 7,405,274.

Thus, the present invention provides the use of an anti-CTGF agent for preparation of a medicament for treatment of chronic allograft rejection, for treatment or prevention of fibrosis in an allograft, for prolonging the survival of an allograft in a recipient, or for reducing transplant-associated hypertrophy in an allograft.

The anti-CTGF agent may be administered alone or in combination with other therapeutics typically used in connection with organ and tissue transplantation, particularly those therapeutics designed or intended to prevent acute transplant rejection, for example, immunosuppressants, corticosteroids, calcineurin inhibitors, antimetabolites, and the like.

### BRIEF DESCRIPTION OF THE DRAWINGS

FIG. 1. Elevated intragraft expression of TGFβ, IL-6, and CTGF in cardiac allografts undergoing CR. TGFβ, IL-6, and CTGF mRNA levels were determined at day 30 post transplant using quantitative real time PCR in syngeneic cardiac grafts, cardiac allografts from recipients treated with anti-CD40L mAb therapy (Anti-CD40L), or cardiac allografts whose recipients were transiently depleted of CD4+ cells (Anti-CD4). Bars represent mean+S.E.M. of 4-9 grafts with expression relative to glyceraldehyde 3-phosphate dehydrogenase (GAPDH) normalized to the syngeneic group.

FIG. 2. Forced expression of TGFβ or CTGF promotes allograft fibrosis. Morphometric analysis of Masson's trichrome staining at day 30 post transplant in cardiac grafts that were left untransduced or transduced with adenoviral vectors encoding β-galactosidase (Adβgal), CTGF (AdCTGF), or TGFβ (AdTGFβ) prior to grafting into syngeneic recipients or allogeneic recipients treated with anti-CD40L. Bars represent the combined mean+S.E.M. of fibrotic area of 10-12 frames of view per heart taken from 5 to 12 different cardiac grafts per group.

FIG. 3. CTGF neutralization ameliorates fibrosis. (A) Mean fibrotic area determined by morphometric analysis of Masson's trichrome staining of cardiac allografts from recipients transiently depleted of CD4+ cells (Anti-CD4) at day 30 post transplant in recipients treated with control IgG or neutralizing anti-CTGF mAb. Bars represent mean+S.E.M. of 10-12 frames of view from each of 6 to 9 hearts. (B) TGFβ, IL-6, and CTGF message levels were determined at day 30 post transplant using quantitative real time PCR in cardiac allografts described in (A). Bars represent mean+S.E.M. of samples taken from 8-12 different cardiac grafts with expression relative to GAPDH normalized against hIgG-treated controls.

FIG. 4. CTGF neutralization ameliorates cardiac hypertrophy in CR grafts. (A) Cardiomyocyte area was quantified from hematoxylin and eosin (H&E) stains of day 30 post transplant cardiac allografts taken from recipients transiently depleted of CD4+ cells (Anti-CD4) and receiving CTGF neutralizing mAb (Anti-CTGF) or control antibodies (hIgG), recipients treated with Anti-CD40L mAb, or naïve, untransplanted hearts from BALB/c mice. Bars represent mean+S.E.M. of area measurements taken from ≧100 cardiomyocytes per heart from 5 (naïve BALB/c and Anti-CD40L), 8 (Anti-CD4+hIgG), or 10 (Anti-CD4+Anti-CTGF) different hearts per group. (B) Intragraft message levels of atrial natriuretic peptide (ANP), a marker of cardiac hypertrophy, were quantified with real time PCR in cardiac grafts from groups in (A) at day 30 post transplant. Bars represent mean+S.E.M. of 8-12 grafts per experimental group (Anti-CD4+hIgG or Anti-CTGF) and 4 grafts per control group (Anti-CD40L and naïve BALB/c mice) with expression relative to GAPDH normalized against the naïve BALB/c hearts.

FIG. 5. CTGF neutralization limits graft infiltration by T cells in CR grafts. (A) Intragraft message levels of T cell receptor β constant region (TCRβ) were quantified at day 30 post transplant with real time PCR as a measure of T cell infiltration of allografts in recipients transiently depleted of CD4+ cells (Anti-CD4) and receiving anti-CTGF mAb or control hIgG antibodies, recipients treated with Anti-CD40L mAb, or naïhearts from BALB/c mice. Bars represent mean+S.E.M. of 8-12 grafts per group with expression relative to GAPDH normalized against the hIgG group. (B) Repopulation of CD4+ cells in the periphery at day 30 post transplant was determined by flow cytometric analysis of splenocytes isolated from graft recipients. Bars represent mean+S.E.M. of the percentage CD4+ cells of the gated cell population in 5 to 7 recipients tested.

### DETAILED DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments, a reference to “an antibody” is a reference to one or more antibodies and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications, which might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D. M. Weir and C. C. Blackwell, eds., 1986, Blackwell Scientific Publications); Sambrook, J. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

### DEFINITIONS

An “allograft” or “allogeneic transplant” is the transplant of an organ (or part of an organ) or tissue from one individual to another individual of the same species with a different genotype. A “syngeneic transplant” is the transplant of an organ or tissue from one individual to another individual of the same species with the same genotype (for example, twins).

The term “antibody” includes whole antibodies and any antigen binding fragment or a single chain thereof. Thus an antibody includes any protein or peptide-containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework (FR) region, or any portion thereof. Antibody includes intact immunoglobulin molecules as well as to fragments thereof, such as Fab, F(ab′)2, and Fv fragments, as well as recombinant, synthetic, and genetically engineered versions thereof, which are capable of binding the epitopic determinant, and include polyclonal and monoclonal antibodies. Anti-CTGF antibodies (i.e., antibodies that bind CTGF or fragments of CTGF) can be prepared using intact CTGF polypeptides or using fragments containing small peptides of interest as the immunizing antigen. The polypeptide or oligopeptide used to immunize an animal (e.g., a mouse, rat, rabbit, chicken, turkey, goat, etc.) can be derived, inter alia, from proteolysis of the CTGF protein, the translation of CTGF mRNA, or synthesized chemically, and can be conjugated to a carrier protein if desired. Commonly used carriers chemically coupled to peptides include, for example, bovine serum albumin, thyroglobulin, and keyhole limpet hemocyanin (KLH). Other methods of selecting antibodies (e.g., phage display) having desired specificities are well known in the art.

The term “antibody” is further intended to encompass antibodies, protease digestion fragments thereof, specified portions and variants thereof, including antibody mimetics or comprising portions of antibodies that mimic the structure and/or function of an antibody or specified fragment or portion thereof, including single chain antibodies and antigen-binding fragments thereof. Examples of antigen-binding fragments of an antibody include, but are not limited to, (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426, and Huston et al. (1988) Proc. Natl. Acad Sci. USA 85:5879-5883). These antibody fragments are obtained using conventional techniques known to those with skill in the art, and the fragments are screened for utility in the same manner as are intact antibodies.

The term “neutralizing antibody” as used herein refers to an antibody, preferably a monoclonal antibody, that is capable of substantially inhibiting or eliminating a biological activity of CTGF. Typically, a neutralizing antibody will inhibit binding of CTGF to a cofactor such as TGFβ, to a CTGF-specific receptor associated with a target cell, or to another biological target.

“Anti-CTGF agent” means any agent, molecule, macromolecule, compound, or composition that inhibits, reduces, or stops the activity, function, production or expression of connective tissue growth factor. The anti-CTGF agent is preferably one that is specific for CTGF and exerts its effect directly and specifically on the CTGF protein or on the CTGF gene or mRNA, rather than a non-specific inhibitor (e.g., a non-specific protease or transcription inhibitor) or an indirect inhibitor (e.g., an inhibitor of an upstream inducer of CTGF). Anti-CTGF agents are well known in the art and are further described herein.

“Anti-CTGF antibody” is an antibody that specifically binds CTGF (i.e, recognizes an epitope of a CTGF polypeptide or fragment). As used herein, “specific binding” refers to antibody binding to a predetermined antigen. Typically, the antibody binds the antigen with a dissociation constant (K_{D}) of 10⁻⁷ M or less, and binds to the predetermined antigen with a K_{D} that is at least 1.5-fold less (preferably at least 2-fold less, more preferably at least 5-fold less) than its K_{D} for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which specifically binds to an antigen”. Anti-CTGF antibodies used in the present invention preferably have a K_{D} for CTGF of 10⁻⁸M or less.

“Connective Tissue Growth Factor” or “CTGF” refers to a protein having amino acid sequences of substantially purified CTGF derived from any species, particularly a mammalian species, including rat, rabbit, bovine, ovine, porcine, murine, equine, and hominid, preferably the human species, and from any source, whether natural, synthetic, semi-synthetic, or recombinant. The sequences of CTGF proteins from numerous species are well known and available from, e.g., the Entrez Nucleotide database from the National Center for Biotechnology Information (NCBI), a division of the National Library of Medicine at the National Institutes of Health. (For example, the human CTGF sequence is Accession No. NP_{—}001892.1; chimpanzee CTGF is Accession No. XP_{—}518744.2; mouse CTGF is Accession No. NP_{—}034347.1; rat CTGF is NP_{—}071602.1. The corresponding CTGF gene and coding sequences are also well known and available in the NCBI database and others.)

The term “fibrosis” refers to abnormal processing of fibrous tissue, or fibroid or fibrous degeneration. Fibrosis can result from various injuries or diseases, and can often result from chronic transplant rejection relating to the transplantation of various organs. Fibrosis typically involves the abnormal production, accumulation, or deposition of extracellular matrix components, including overproduction and increased deposition of, for example, collagen and fibronectin. “Fibrosis” is used herein refer to any excess production or deposition of extracellular matrix proteins that occurs in allografts as a component of the chronic rejection.

A “therapeutically effective amount” is the dose needed to effectively treat the physiological effects of chronic allograft rejection.

By “transplant” or “transplantation” is intended the relocation, typically by surgical procedure, of an organ or tissue from one body to another (or from a donor site on the patient's own body to a recipient site), generally for the purpose of replacing the recipient's damaged or failing organ or tissue with a functional one from the donor site. The transplanted organ or tissue is a “graft.” Grafts can be autografts (donor and recipient are same individual), isografts or homografts (donor and recipient of same species and same genotype), allografts (donor and recipient of same species but different genotype) or xenografts (donor and recipient of different species).

By “transplant-associated hypertrophy” is intended the pathological occurrence of hypertrophy in transplanted organs and tissues. Such transplant-associated hypertrophy is particularly seen in cardiac transplants.

“Transplant rejection” or “allograft rejection” occurs when the transplanted organ or allograft is not accepted by the recipient's body. Both acute rejection and chronic rejection are typical in allograft transplantation. Organ transplants evoke a variety of immune responses in the host. In acute rejection, the graft is initially invaded by the host's mononuclear cells (macrophages, lymphocytes, and monocytes). If these cells perceive antigenic differences in the graft, they will process and present the antigen to a T-lymphocyte and activate it in an antigen specific manner. This T-cell then stimulates an immune response, which usually is a combination of cellular (T-cell mediated) and humoral (B-cell mediated) responses. These reactions appear to be the primary cause of the early acute rejection that may occur within the first six weeks after transplant. If untreated, acute rejection is a rapid and severe process that causes destruction of the transplant within a few days. In general, if the patient has not had an acute rejection within the first six weeks or if the patient has had a rejection that has been successfully treated, it is unlikely that a separate episode of acute rejection will occur provided that the patient continues standard immunosuppressive therapy. “Chronic allograft rejection” (or “chronic rejection” or “CR”), on the other hand, is a gradual process of deterioration and failure that occurs later in the life of the transplant, from several months to several years. Chronic rejection, which manifests as progressive and generally irreversible damage to the graft from attack due to host immune responses, is the leading cause of organ transplant loss after the first postoperative year. A significant proportion of grafts, regardless of type, deteriorate and fail within the first several months or years after transplant despite administration of maintenance immunosuppression. Two components associated with chronic rejection are luminal occlusion and interstitial fibrosis. Although the pace of graft destruction varies from patient to patient, essentially all heart or kidney transplants of cadaveric origin eventually succumb to chronic rejection. This persistent rate of decline of long-functioning grafts has remained constant over time, despite improvements in immunosuppressive therapy and in overall care for graft recipients.

The etiology of CR is not fully understood, however, multiple factors have been associated with its onset and progression, especially transforming growth factor-β (TGFβ). TGFβ overexpression is linked with chronic rejection (Csencsits K et al., Am J Transplant 2006 6(5 Pt 1):959-966; Jain S et al., Transplantation 2000 69(9):1759-1766) and may negatively impact graft survival through chemotactic and pro-fibrotic effects (Li et al. Annu Rev Immunol 2006 24:99-146.). However, in addition to its deleterious fibrotic effects on the graft, TGFβ's immunosuppressive and anti-proliferative functions may be indispensable for graft and host survival (Blobe G C et al. N Engl J Med 2000 342(18):1350-1358). For example, TGFβ plays a critical role in the induction and function of T regulatory cells (Treg), which are believed to contribute to graft acceptance (Wood K J et al. Nat Rev Immunol 2003 3(3):199-210; Yong Z et al. Transpl Immunol 2007 17(2):120-129; Walsh P T et al. J Clin Invest 2004 114(10):1398-1403). In addition, TGFβ inhibits T and B cell proliferation (Li et al. Annu Rev Immunol 2006 24:99-146) and represses cancers of epithelial cell origin (Brattain M G et al. Curr Opin Oncol 1996 8(1):49-53). These opposing effects make TGFβ a suboptimal target for CR treatments and have prompted investigation into the downstream mediators of TGFβ in CR pathology. Identifying downstream mediators of CR may facilitate the development of therapeutics that negate the fibrosis-inducing activity of TGFβ while sparing its anti-inflammatory and anti-proliferative effects.

One such downstream mediator, known to be induced by TGFβ in multiple cell types (Leask A and Abraham DJ. Biochem Cell Biol 2003 81(6):355-363), including cardiac myocytes and fibroblasts (Chen M M et al. J Mol Cell Cardiol 2000 32(10):1805-1819), is connective tissue growth factor (CTGF). CTGF is a 36 kD, cysteine-rich, heparin binding, secreted glycoprotein originally isolated from the culture media of human umbilical vein endothelial cells. (See e.g., Bradham et al. (1991) J Cell Biol 114:1285-1294; Grotendorst and Bradham, U.S. Pat. No. 5,408,040.) CTGF belongs to the CCN (TGF, Cyr61, Nov) family of proteins (secreted glycoproteins), which includes the serum-induced immediate early gene product Cyr61, the putative oncogene Nov, the ECM-associated protein FISP-12, the src-inducible gene CEF-10, the Wnt-inducible secreted protein WISP-3, and the anti-proliferative protein HICP/rCOP (Brigstock (1999) Endocr Rev 20:189-206; O'Brian et al. (1990) Mol Cell Biol 10:3569-3577; Joliot et al. (1992) Mol Cell Biol 12:10-21; Ryseck et al. (1990) Cell Growth and Diff 2:225-233; Simmons et al. (1989) Proc Natl Acad Sci USA 86:1178-1182; Pennica et al. (1998) Proc Natl Acad Sci USA, 95:14717-14722; and Zhang et al. (1998) Mol Cell Biol 18:6131-6141.) CTGF is also referred to as CCN2 or IGFBP8. CCN proteins are characterized by conservation of 38 cysteine residues that constitute over 10% of the total amino acid content and give rise to a modular structure with N- and C-terminal domains. The modular structure of CTGF includes conserved motifs for insulin-like growth factor binding protein (IGF-BP) and von Willebrand's factor (VWC) in the N-terminal domains (Domains 1 and 2, respectively), and thrombospondin (TSP1) and a cysteine-knot motif in the C-terminal domains (Domains 3 and 4, respectively) (see, DeWinter et al. (2008) Growth Factors 26:80-91 for a review).

CTGF plays an important role in the development of connective tissue as well as the formation of scar tissue (de Winter P et al. Growth Factors 2008; 26(2):80-91; Bonniaud P et al. Am J Respir Cell Mol Biol 2004; 31(5):510-516), and is upregulated in multiple fibrotic disorders, including CR of cardiac and kidney grafts (Csencsits K et al. Am J Transplant 2006 6(5 Pt 1):959-966; Cheng 0 et al. Am J Transplant 2006 6(10):2292-2306; Yuan Y C et al. J Formos Med Assoc 2009 108(3):240-246; Daniels A et al. Acta Physiol (Oxf) 2009 195(3):321-338). CTGF mediates multiple pro-fibrotic effects ascribed to TGFβ including increased extra cellular matrix production, fibroblast proliferation, and enhancement of adhesive responses (Daniels A et al. Acta Physiol (Oxf) 2009 195(3):321-338).

In accordance with the present invention there is provided a method for treating chronic allograft rejection in a subject having received or about to receive an allograft, wherein the method comprises administering a therapeutically effective amount of an anti-CTGF agent to said subject. The method optionally comprises the additional step of determining the extent of the allograft rejection subsequent to the administration of the anti-CTGF agent. The extent of the allograft rejection can be determined by any number of methods that are well known in the art, some of which are described herein, for example by assessing the loss of function of the transplanted organ, by tissue biopsy of the allograft and determination of the mean fibrotic area of the tissue, or by analysis of any of a number of well known biomarkers for chronic rejection. In most cases the parameters measured to assess the extent of CR will vary with the specific type of transplanted organ/tissue. These parameters are well known in the art and some particular examples are discussed herein. The extent of the allograft rejection is preferably determined at a sufficient time after administration of the anti-CTGF agent to allow manifestation of therapeutic effect of the agent. The extent of the chronic rejection may also be determined at a time prior to administration of the agent for determination of subjects in need of treatment for CR and/or for a baseline value for comparison with normal or post-treatment samples. The subject is any individual that has received or is about to receive an allograft by transplantation. Preferably, the subject is a mammalian subject, including but not limited to, human, non-human primate, sheep, horse, cattle, goat, pig, dog, cat, rabbit, guinea pig, hamster, rat, mouse, as well as the organs and tissues derived or originating from these hosts; most preferably, the subject is a human subject.

In another embodiment, the present invention provides a method for prolonging the survival of an allograft in an allograft recipient, wherein the method comprises administering a therapeutically effective amount of an anti-CTGF agent to said recipient. The method optionally comprises the additional step of comparing the post-transplant survival rate of said allograft to a standard post-transplant survival rate. “Survival of the allograft” means the continued functioning of the transplanted organ or tissue in the recipient at a level acceptable for maintenance of the recipient. Survival of the allograft is determined by any of a number of methods that are conventional and acceptable in the transplantation arts. The standard post-transplant survival rate is the percentage of allografts that survive until certain benchmark time points (e.g., a 1-year survival rate, a 3-year survival rate, a 5-year survival rate, etc.). The standard survival rates for many different organ and tissue transplants are compiled by various organizations and are well known to those of ordinary skill in the transplantation arts (e.g., the Organ Procurement and Transplantation Network in the U.S. which was established in 1984 by the National Organ Transplant Act; the International Society for Heart and Lung Transplantation (ISHLT)). The allograft recipient is an individual that has received an allograft by transplantation. The allograft recipient is preferably a mammal, including for example, human, non-human primate, sheep, horse, cattle, goat, pig, dog, cat, rabbit, guinea pig, hamster, rat, mouse; most preferably, the allograft recipient is a human.

In a separate embodiment, the invention provides a method of reducing or preventing fibrosis in an allograft, wherein the method comprises administering a therapeutically effective amount of an anti-CTGF agent to said allograft. The agent can be administered directly to the allograft, either before or subsequent to transplantation or both, or can be administered indirectly by administration of the agent to the allograft recipient. The method optionally comprises the additional step of determining the amount of fibrosis in the allograft subsequent to administration of the anti-CTGF agent. The amount of fibrosis in the allograft is preferably determined at a sufficient time after administration of the anti-CTGF agent to allow manifestation of therapeutic effect of the agent. The amount of fibrosis in the allograft may also be determined at a time prior to administration of the agent for determination of subjects in need of treatment for allograft fibrosis and/or for a baseline value for comparison with normal or post-treatment samples. The amount of fibrosis in the allograft can be determined by any of a number of conventional methods, e.g., histological examination of biopsy tissue from the allograft, detection of markers of fibrosis (e.g., increased level of collagen, CTGF, TGF-β, etc.).

In another embodiment, the invention provides a method of reducing or preventing transplant-associated hypertrophy in an allograft, wherein the method comprises administering a therapeutically effective amount of an anti-CTGF agent to said allograft. The agent can be administered directly to the allograft, either before or subsequent to transplantation or both, or can be administered indirectly by administration of the agent to the allograft recipient. The method optionally comprises the additional step of determining the amount of hypertrophy in said allograft subsequent to the administration of the ant-CTGF agent. The amount of hypertrophy in the allograft may also be determined at a time prior to administration of the agent for comparison. The amount of hypertrophy in the allograft can be determined by any of a number of conventional methods, e.g., echocardiography, measurement of cardiomyocyte size, biomarkers (e.g., ANP level). In a particular embodiment, the invention provides a method of reducing or preventing cardiac hypertrophy in a cardiac allograft.

For any of the methods of the present invention, the allograft can be any type of organ or tissue that is capable of being transplanted and that is subject to chronic rejection. Such organs and tissues include heart, heart valves, liver, lung, kidney, intestine, skin, eye, cornea, pancreas, ligament, tendon, and bone, composite tissue transplants and multiple organ transplants. In a preferred embodiment the allograft is selected from heart, heart valves, liver, lung, and kidney; in a more preferred embodiment, the allograft is selected from heart, heart valves, liver, and lung. Most preferably, the allograft is heart.

In general, for any of the methods of the present invention the anti-CTGF agent may be administered at the time of the transplantation procedure, immediately following the transplant procedure (e.g., within 7 days following the transplantation procedure), or at a time subsequent to the transplant procedure, or at some or all of these times. When administration of the agent is at a time subsequent to the transplant procedure, the administration typically begins at a time between 7 and 21 days post-transplant to allow some period of recovery and wound healing for the transplant recipient prior to beginning treatment with the anti-CTGF agent. Alternatively, the administration of anti-CTGF agent can be delayed until a later time, including up until the onset of CR and beyond. Alternatively, or in addition, the anti-CTGF agent can be administered prior to a transplantation procedure to a subject that is about to receive an allograft, or in some cases, to the organ or tissue to be transplanted. In general for this embodiment, the agent is administered just prior to and in preparation for the transplantation procedure, typically no more than 24 hours prior to the transplantation procedure. One of ordinary skill in the transplantation arts is competent to determine the optimum time for administration of the anti-CTGF agent.

### Chronic Rejection

Chronic rejection develops months to years after acute rejection episodes have subsided. Chronic rejections are both antibody- and cell-mediated. The use of immunosuppressive drugs and tissue-typing methods has increased the survival of allografts in the first year, but chronic rejection is not prevented in most cases. Chronic rejection appears as fibrosis and scarring in all transplanted organs, but the specific histopathological picture depends on the organ transplanted. In heart transplants, chronic rejection typically manifests as accelerated coronary artery disease or transplant-associated vasculopathy. In transplanted lungs, it manifests as bronchiolitis obliterans syndrome. In liver transplants, chronic rejection is most often characterized by the vanishing bile duct syndrome. In kidney recipients, chronic rejection (called chronic allograft nephropathy) typically manifests as fibrosis and glomerulopathy. The following factors increase the risk of chronic rejection: a previous episode of acute rejection, inadequate immunosuppression, initial delayed graft function, donor-related factors (e.g., old age, hypertension), reperfusion injury to organ, long cold ischemia time, recipient-related factors (e.g., diabetes, hypertension, hyperlipidemia), and posttransplant infection (e.g., cytomegalovirus).

In accordance with the present invention, chronic allograft rejection is characterized by an acute or chronic diminution in the physiological function of a transplanted organ or tissue. Such diminution in function can be measured by biological factors specific to the organ transplanted.

Diagnosis of cardiac allograft rejection is usually made with the use of endomyocardial biopsy, either done on a surveillance basis or in response to clinical deterioration. Biopsy surveillance is performed on a regular basis in most programs for the first year post-operatively and for the first 5 years in many programs. Despite having young donor hearts, cardiac allograft recipients are prone to develop coronary artery disease (CAD). This CAD is generally a diffuse, concentric and longitudinal process that is quite different from ordinary atherosclerotic CAD which is more focal and often eccentric. Typical parameters for assessment of heart transplant rejection include increased cardiac vessel disease post-transplant, and increased graft intimal hyperplasia, which independently or taken together are indicators of graft rejection.

For kidney grafts, often after a long period of satisfactory and stable function, the recipient begins to develop characteristic manifestations of glomerulosclerosis and nephrosclerosis, e.g., progressive proteinuria, hypertension, and declining kidney function. Histological findings are tubular atrophy, parenchymal fibrosis and chronic arteritis. A system of assessing the extent of chronic rejection based on histological determinations, the Banff classification (Racusen et al. Kidney Intl. 1999 55:713), is a suitable method for assessing the extent of chronic allograft nephropathy. In some cases, renal transplant rejection may present only as a rise in serum creatinine levels. Doppler ultrasonic or magnetic resonance angiography may be useful in ascertaining changes in the renal vasculature and in renal blood flow, even in the absence of urinary flow. Diagnostic ultrasound is the procedure of choice to rule out urinary obstruction or to confirm the presence of perirenal collections of urine, blood or lymph. In other cases renal biopsy may be the only way to diagnose rejection. For example, for kidney transplant rejection assessment, increased glomerular atrophy, intimal thickening, hyalinization, tubular atrophy, interstitial fibrosis, lymphocyte infiltration and cortical scarring independently or taken together are indicators of graft rejection.

In liver transplants, chronic rejection is characterized by progressive cholestasis, focal parenchymal necrosis, mononuclear infiltration, vascular lesions (intimal fibrosis, subintimal foam cells, fibrinoid necrosis) and fibrosis. This process may be reflected as ductopenia. Clinically, the recipient may develop relatively asymptomatic jaundice followed by increasing levels of serum bilirubin, alkaline phosphatase, and glutamyl transpeptidase. Unlike acute early rejection in which bile ducts become distorted by infiltration of inflammatory cells and the epithelial cell changes involve both nuclei and organelles, the lesions in chronic rejection are characteristic focal destruction of the bile ducts, particularly involving the smaller interlobular bile ducts and progressing in some cases to the disappearance of these ducts. Arteriosclerotic-like changes occur in blood vessels with arterial wall thickening causing vascular insufficiency. The larger branches of the hepatic artery often develop graft arteriosclerosis, myointimal proliferation, foam cell deposition and intimal inflammation. Histologically, obliterative arteriopathy is characterized by T-cell infiltration of the vessel walls, suggesting that the lesion is immunological in nature.

In lung transplants, chronic rejection is the main impediment to better medium-term survival rates. Clinically, chronic rejection is a form of graft dysfunctions that is synonymous with bronchiolitis obliterans syndrome (BOS). BOS is characterized physiologically by airflow limitation and pathologically by bronchiolitis obliterans. BOS is generally diagnosed based on a sustained decrement (20%) in the FEV₁. A smaller decline in the FEV₁ (forced expiratory volume in 1 s) or a decrease in the FEF_{25-75%} (forced expiratory flow between 25 and 75% of the vital capacity) may also presage BOS. Histologically, the condition manifests with bronchiolar epithelial ulceration, peribronchial inflammatory infiltrate, and intraluminal plugs of mucus, with necrotic cells and granulation tissue. As the chronic process evolves, infiltrates decrease and the bronchiolar openings become progressively obliterated by fibrosis.

In addition to determination of functional and structural assessment of the transplanted organ/tissue, certain markers correlated with chronic rejection are known and can be measured to evaluate the progress or extent of rejection. Such chronic rejection markers include increased expression of collagen, elastin (characteristic of neointimal development) CTGF (e.g., Cheng et al., 2006 Amer. J. Transplantation 6:2292), TGF-β (e.g., Csencsits et al. 2006 Amer. J. Transplantation 6:959), ribosomal L7, β-transducin, 1-TRAF, lysyl tRNA transferase (see, U.S. Pat. No. 7,132,245), soluble CD30 levels (Golocheikine et al. 2008 Transpl. Immunol. 18:260). Panels of biomarkers, or signatures, of chronic rejection have been identified (see, for example, Quintana et al. J. Am. Soc. Nephrol. 2009 20:428; Brouard et al. Proc. Natl Acad Sci. 2007 104:15448; Intl. Pub. No. WO2009/060035; Intl Pub. No. WO2004/018710).

### Anti-CTGF Agents

In any of the methods described above, it is particularly contemplated that the anti-CTGF agent may be a polypeptide, polynucleotide, or small molecule; for example, an antibody that binds to CTGF, a CTGF antisense molecule, miRNA, ribozyme or siRNA, a small molecule chemical compound, etc. In particular, the present invention contemplates that inhibiting CTGF can be accomplished by any of the means well-known in the art for modulating the expression or activity of CTGF. Use of anti-CTGF agent, for example, a human monoclonal antibody directed against CTGF, is preferred, although any method of inhibiting expression of the gene encoding CTGF, inhibiting production of CTGF, or inhibiting activity of CTGF is contemplated by the present invention.

Exemplary antibodies for use in the methods of the present invention are described, e.g., in U.S. Pat. No. 5,408,040; International Publication No. WO 99/07407; International Publication No. WO 99/33878; and International Publication No. WO 00/35936. Preferably, the anti-CTGF antibody for use in the method is a monoclonal antibody. Preferably the antibody is a neutralizing antibody. The antibody produced by ATCC Accession No. PTA-6006 cell line is a preferred such antibody. Exemplary monoclonal anti-CTGF antibodies for use in the methods of the present invention include CLN1 or mAb1 described in U.S. Pat. No. 7,405,274, which reference is incorporated by reference herein in its entirety. Variants of CLN1 that retain the binding and neutralization functions characteristic of CLN1 are also useful in the present invention. Such variants typically retain the variable regions of the heavy and/or light chain of the original neutralizing antibody, or minimally the complementarity determining regions (CDR) of heavy and light chains, and may contain substitutions and/or deletions in the amino acid sequences outside of those variable regions. Fragments and engineered versions of the original neutralizing antibody, e.g., Fab, F(ab)2, Fv, scFV, diabodies, triabodies, minibodies, chimeric antibodies, humanized antibodies, etc. are likewise useful in the method of the present invention. Such antibodies, or fragments thereof, can be administered by various means known to those skilled in the art. For example, antibodies are often injected intravenously, intraperitoneally, or subcutaneously.

Small molecule inhibitors of CTGF expression and/or activity have also been described; for example, International Publication No. WO 96/38172 identifies modulators of cAMP such as cholera toxin and 8Br-cAMP as inhibitors of CTGF expression. Therefore, compounds identified as, e.g., prostaglandin and/or prostacyclin analogs such as Iloprost (see, e.g., International Publication No. WO 00/02450; Ricupero et al. (1999) Am J Physiol 277:L1165-1171; also, see Ertl et al. (1992) Am Rev Respir Dis 145:A19), and potentially phosphodiesterase IV inhibitors (see, e.g., Kohyama et al. (2002) Am J Respir Cell Mol Biol 26:694-701), may be used to modulate CTGF expression. Also, inhibitors of serine/threonine mitogen activated protein kinases, particularly p38, cyclin-dependent kinase, e.g. CDK2, and glycogen synthase kinase (GSK)-3 have also been implicated in decreased CTGF expression. (See, e.g., Matsuoka et al. (2002) Am J Physiol Lung Cell Mol Physiol 283:L103-L112; Yosimichi et al. (2001) Eur J Biochem 268:6058-6065; International Publication No. WO 01/38532; and International Publication No. WO 03/092584.) Such compounds can be formulated and administered according to established procedures within the art.

Further, polynucleotide inhibitors of CTGF, including small interfering ribonucleic acids (siRNAs), micro-RNAs (miRNAs), ribozymes, and antisense sequences may be used in the present methods to inhibit expression and/or production of CTGF. (See, e.g., Kondo et al. (2000) Biochem Biophys Res Commun 278:119-124.) Such techniques are well-known to those of skill in the relevant art. Polynucleotide inhibitors that target CTGF expression have been described and utilized to reduce CTGF expression in various cell types. (See, e.g., International Publication No. WO 96/38172; International Publication No. WO 00/27868; International Publication No. WO 00/35936; International Publication No. WO 03/053340; Kothapalli et al. (1997) Cell Growth Differ 8(1):61-68; Shimo et al. (1998) J Biochem (Tokyo) 124(1):130-140; Uchio et al. (2004) Wound Repair Regen 12:60-66; Guha et al. FASEB J. 2007 21:3355; U.S. Pat. No. 6,358,741; U.S. Pat. No. 6,965,025; U.S. Pat. No. 7,462,602; US Application Publication No. 2008/0070856; US Application Publication No. 2008/0176964) CTGF antisense constructs and other types of polynucleotide inhibitors of CTGF can be used to reduce expression of CTGF and thereby ameliorate or prevent the chronic allograft rejection. Such constructs can be designed using appropriate vectors and expressional regulators for cell- or tissue-specific expression and constitutive or inducible expression. Such genetic constructs can be formulated and administered according to established procedures within the art.

Accordingly, in certain embodiments of the present invention, the anti-CTGF agent is an anti-CTGF antibody. In a preferred embodiment, the anti-CTGF antibody is a monoclonal antibody. In a particularly preferred embodiment, the antibody is a neutralizing antibody. In another preferred embodiment, the antibody is a human or humanized antibody to CTGF. In a more preferred embodiment, the antibody recognizes an epitope within domain 2 of CTGF. In a particular embodiment, the antibody is CLN1, as described in U.S. Pat. No. 7,405,274. In a particular embodiment, the antibody is the antibody produced by ATCC Accession No. PTA-6006 cell line, as described in U.S. Pat. No. 7,405,274. In another embodiment, the agent is a small molecule. In another embodiment, the agent is a polynucleotide inhibitor of CTGF. In particular embodiments, polynucleotide inhibitor is a CTGF antisense oligonucleotide, a CTGF miRNA, a CTGF ribozyme or CTGF siRNA.

As further described herein, the anti-CTGF agent can be administered alone or in combination with other therapeutics, particularly therapeutics for treatment and prevention of acute allograft rejection. The present invention contemplates the use of the present methods in combination with other therapies. In one embodiment, the method is used in combination with another therapy, e.g., to further augment therapeutic effect on certain pathological events, etc. The two treatments may be administered at the same time or consecutively, e.g., during a treatment time course or following transplantation and rejection (acute or chronic). Current therapeutic practice for organ and tissue transplantation includes the use of corticosteroids (e.g., prednisone), antimetabolites (e.g., azathioprine, mycophenolate mofetil), calcineurin inhibitors (e.g., tacrolimus, cyclosporine), Sirolimus (rapamycin), thymoglobulin, OKT3, daclizumab, basiliximab, and/or alemtuzumab. Use of any of these therapeutic agents in combination with the anti-CTGF agents for use in methods of the present invention is specifically contemplated, including use of an anti-CTGF agent for the preparation of a medicament for treating chronic allograft rejection in a subject having received or about to receive an allograft, use of an anti-CTGF agent for the preparation of a medicament for prolonging the survival of an allograft in an allograft recipient, use of an anti-CTGF agent for the preparation of a medicament for reducing or preventing fibrosis in an allograft in a subject having received, and use of an anti-CTGF agent for the preparation of a medicament for reducing or preventing transplant-associated hypertrophy in an allograft in an allograft recipient.

For anti-CTGF antibody agents, depending on the type and severity of the disease, about 0.015 to 50 mg of antibody/kg of patient weight is an initial candidate dosage for administration to the patient, whether, for example, by one or more separate administrations, or by continuous infusion. Typically, a dose of between 0.5 and 15 mg/kg is used; preferably, a dose of between 1 mg/kg and 5 mg/kg is used. For repeated administrations over several days or longer, depending on the condition, the treatment is repeated until a desired suppression of disease symptoms (e.g., fibrosis or hypertrophy) occurs. However, other dosage regimens may be useful and are not excluded from the present invention.

### Pharmaceutical Formulations and Routes of Administration

The anti-CTGF agents used in the method of the present invention can be delivered directly or in pharmaceutical compositions containing excipients, as is well known in the art. Present methods of treatment can comprise administration of an effective amount of a compound or composition of the present invention to a subject who has received or is about to receive a transplant. Subjects specifically intended for treatment with the compositions and methods of the present invention include humans, as well as non-human primates, sheep, horses, cattle, goats, pigs, dogs, cats, rabbits, guinea pigs, hamsters, rats, and mice, as well as the organs or tissue, derived or originating from these hosts. In a preferred embodiment, the subject is a mammalian subject, and in a most preferred embodiment, the subject is a human subject.

An effective amount of compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences, supra; and Goodman and Gilman's The Pharmacological Basis of Therapeutics, 10^{th} Ed. (2001), Hardman, Limbird, and Gilman, eds. MacGraw Hill Intl)

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred. In the methods of the present invention preferred routes of administration included intraperitoneal, intravenous and subcutaneous.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art, and include those listed in various pharmacopoeias. (See, e.g., USP, JP, EP, and BP, FDA web page (www.fda.gov), Inactive Ingredient Guide 1996, and Handbook of Pharmaceutical Additives, ed. Ash; Synapse Information Resources, Inc. 2002.)

Pharmaceutical dosage forms of an anti-CTGF agent for use in the method of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include, fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methan and ethan, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and, can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound, sucrose or sodium chloride as a tonicity agent, for example, the buffer contains phosphate or histidine. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

The therapeutically effective amount is the amount of the agent or pharmaceutical composition that will elicit the biological or medical response of a tissue, system, animal, or human that is being sought by the researcher, veterinarian, medical doctor, or other clinician, e.g., a reduction in chronic allograft rejection, a prolongation of the survival of an allograft, the prevention or reduction of transplant-associated hypertrophy associated with an allograft, etc. For compositions useful for the present methods of treatment, a therapeutically effective amount can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays. Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD50/ED50. Agents that exhibit high therapeutic indices are preferred.

Dosages preferably fall within a range of circulating concentrations that includes the ED50 with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects, e.g., reduction in chronic allograft rejection, reduction or prevention of fibrosis, a prolongation of the survival of an allograft, the prevention or reduction of hypertrophy associated with an allograft etc, i.e., minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack, or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### EXAMPLES

The invention will be further understood by reference to the following examples, which are intended to be purely exemplary of the invention. These examples are provided solely to illustrate the claimed invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications are intended to fall within the scope of the appended claims.

### Methods and Materials

Mice. Female C57BL/6 (H-2^{b}) and BALB/c (H-2^{d}) mice were obtained from Charles River Laboratories (Raleigh, N.C.) and were kept under micro-isolator conditions. The use of mice for these studies was reviewed and approved by the University of Michigan's Committee On The Use And Care Of Animals.

Vascularized cardiac transplantation. Heterotopic cardiac transplantation was performed as described (Corry R J et al. Transplantation 1973 16(4):343-350). Briefly, the aorta and pulmonary artery of the donor heart were anastomosed end-to-side to the recipient's abdominal aorta and inferior vena cava, respectively. Upon perfusion with the recipient's blood, the transplanted heart resumes contraction. Graft function is monitored by abdominal palpation.

In vivo mAb administration. Anti-CD4 (hybridoma GK1.5, obtained from American Type Culture Collection, Manassas, Va.), and anti-CD40L (hybridoma MR1, kindly provided by Dr. Randy Noelle, Dartmouth College). Allograft recipients were transiently depleted of CD4+ cells by i.p. injection of 1 mg of anti-CD4 mAb on days −1, 0, and 7 post transplant (Csencsits K et al. Am J Transplant 2006 6(5 Pt 1):959-966). For inductive anti-CD40L therapy, allograft recipients were injected i.p. with 1 mg of anti-CD40L on days 0, 1, and 2 post transplant (Csencsits K et al Am J Transplant 2006 6(5 Pt 1):959-966). Anti-IL-6 mAb or control rat IgG (Sigma, St. Louis, Mo.) was administered by i.p. injection of 1 mg on days −1, 1, and 3 and weekly thereafter (Burrell B E et al. J Immunol 2008 181(6):3906-3914). Allograft recipients treated with anti-CTGF mAb (CLN1) or control human IgG (Sigma, St. Louis, Mo.) received 0.5 mg i.p. twice weekly beginning on day 7 posttransplant.

Adenoviral-mediated transduction of cardiac allografts. Transduction was performed as previously described (Csencsits K et al. Am J Transplant 2006 6(5 Pt 1):959-966; Chan S Y et al. Transplantation 2000 70(9):1292-1301; Chan S Y et al. Nat Med 1999 5(10):1143-1149). Briefly, cardiac allografts were perfused via the aorta with 5×10⁸ pfu of E1/E3 deleted adenoviral vectors encoding the active form of human TGFβ1 (AdTGFβ) (Csencsits K et al. Am J Transplant 2006 6(5 Pt 1):959-966; Chan S Y et al. Transplantation 2000 70(9):1292-1301), human CTGF (AdCTGF) (Haberberger T C et al. Gene Ther 2000 7(11):903-909), or beta-galactosidase (Adβgal) (Csencsits K et al. Am J Transplant 2006 6 (5 Pt 1):959-966; Chan S Y et al. Transplantation 2000 70(9):1292-1301; Chan S Y et al. Nat Med 1999 5 (10):1143-1149). Following perfusion, donor grafts were placed in iced Ringer's solution for 1 hour prior to transplantation. Previous studies with Adβgal have revealed a patchy distribution of transgene expression by both cardiac and vascular cells (Chan S Y et al. Nat Med 1999 5(10):1143-1149).

Morphometric analysis of cardiac allograft fibrosis and hypertrophy. Graft fibrosis was quantified by morphometric analysis of Masson's trichrome stained sections using iPLab software (Scanalytics Inc., Fairfax, Va.). Masson's trichrome is a stain for collagen fibers. Mean fibrotic area was calculated from 10 to 12 areas per heart section analyzed at 200× magnification. To quantify cardiomyocyte area as a measure of hypertrophy, digital outlines were drawn around at least 80 cardiomyocytes from views of H&E stained sections at 200× magnification. Areas within outlines were quantified using SCION IMAGE Beta 4.0.2 software (Scion Corporation, Frederick, Md.) to measure cardiomyocyte cell size (Nozato T et al. Jpn Circ J 2000 64(8):595-601). A minimum of 8 hearts were analyzed per group for both analysis techniques.

Quantitative real time PCR. Graft RNA was isolated by homogenizing tissues in TRIzol reagent (Invitrogen, Carlsbad, Calif.) as per manufacturer's protocol. Five μg of total RNA were reverse transcribed using Oligo dT, dNTPs, MMLV-RT (Invitrogen, Carlsbad, Calif.), RNAsin (Promega, Madison, Wis.) in PCR Buffer (Roche, Indianapolis, Ind.). Resulting cDNA was purified by a 1:1 extraction with phenol/chloroform/isoamyl (25:24:1) then precipitated in one volume 3M NaOAc and two volumes absolute ethanol. Levels of atrial natriuretic peptide (ANP), CTGF, IL-6, TGFβ, IL-17, and T cell receptor constant region (TCRβ) message were determined by quantitative real time PCR using iQ SYBR master mix (Bio-Rad, Hercules, Calif.) in a Rotor-Gene 3000 thermocycler (Corbett Life Science, San Francisco, Calif.). Expression levels were determined relative to GAPDH using the Rotor-Gene Comparative Concentration utility. Primer sequences were as follows:

| | |
|---|---|
| ANP (Nppa) forward | |
| 5′ GGAGGTCAACCCACCTCTG 3′ | (SEQ ID NO: 1) |
| ANP (Nppa) reverse | |
| 5′ GCTCCAATCCTGTCAATCCTAC 3′ | (SEQ ID NO: 2) |
| CTGF (ctgf) forward | |
| 5′ GGAAAACATTAAGAAGGGCAAAA 3′ | (SEQ ID NO: 3) |
| CTGF (ctgf) reverse | |
| 5′ CCGCAGAACTTAGCCCTGTA 3′ | (SEQ ID NO: 4) |
| GAPDH (Gapdh) forward | |
| 5′ CTGGTGCTGAGTATGTCGTG 3′ | (SEQ ID NO: 5) |
| GAPDH (Gapdh) reverse | |
| 5′ CAGTCTTCTGAGTGGCAGTG 3′ | (SEQ ID NO: 6) |
| IL-6 (Il6) forward | |
| 5′ CGTGGAAATGAGAAAAGAGTTGT 3′ | (SEQ ID NO: 7) |
| IL-6 (Il6) reverse | |
| 5′ TCCAGTTTGGTAGCATCCATC 3′ | (SEQ ID NO: 8) |
| TGFβ (Tgfb 1) forward | |
| 5′ CCTGAGTGGCTGTCTTTTGAC 3′ | (SEQ ID NO: 9) |
| TGFβ (Tgfb 1) reverse | |
| 5′ CCTGTATTCCGTCTCCTTGGT 3′ | (SEQ ID NO: 10) |
| IL-17 (Il17a) forward | |
| 5′ GGACTCTCCACCGCAATGA 3′ | (SEQ ID NO: 11) |
| IL-17 (Il17a) reverse | |
| 5′ GACCAGGATCTCTTGCTGGA 3′ | (SEQ ID NO: 12) |
| TCRβ (Tcrb-C) forward | |
| 5′ CTGCCAAGTGCAGTTCCAT 3′ | (SEQ ID NO: 13) |
| TCRβ (Tcrb-C) reverse | |
| 5′ GGCCTCTGCACTGATGTTCT 3′ | (SEQ ID NO: 14) |

Flow cytometry. Splenocytes were labeled with FITC-conjugated anti-CD3, PE-conjugated anti-CD4, and CY-conjugated anti-CD8 (PharMingen San Jose, Calif.). Cell analyses were performed on cells gated using forward vs. side scatter using a Becton Dickinson FACScan (San Jose, Calif.).

Statistical analysis. Statistical significance was calculated using an unpaired t-test with Welch's correction. p values ≦1.05 were considered statistically significant.

### Example 1

### Chronic Allograft Rejection Mouse Model

In the mouse vascularized cardiac allograft model, BALB/c allografts in C57BL/6 recipients receiving anti-CD40L mAb continue to function for >60 days post transplant and do not develop CR (Csencsits K et al. Am J Transplant 2006 6(5 Pt 1):959-966). In contrast, BALB/c allografts in C57BL/6 recipients transiently depleted of CD4+ cells by anti-CD4 antibody treatment develop CR as CD4+ cells begin to repopulate the periphery between 3 and 4 weeks following initial depletion (Csencsits K et al. Am J Transplant 2006 6(5 Pt 1):959-966; Bishop D K et al. Transplantation 1994; 58(5):576-584; Piccotti J R et al. Transplantation 1999 67(12):1548-1555; Csencsits K et al. Am J Transplant 2008 8(8):1622-1630). Previous echocardiographic and histologic analysis revealed that day 30 post transplant represents a critical point in this CR model as extensive graft hypertrophy and fibrosis are present at this time and are followed by degradation of cardiac contractility. Therefore, grafts were assessed at day 30 post transplant in these studies.

### Example 2

### Elevated Intragraft TGFβ, IL-6, and CTGF Expression Correlate with CR

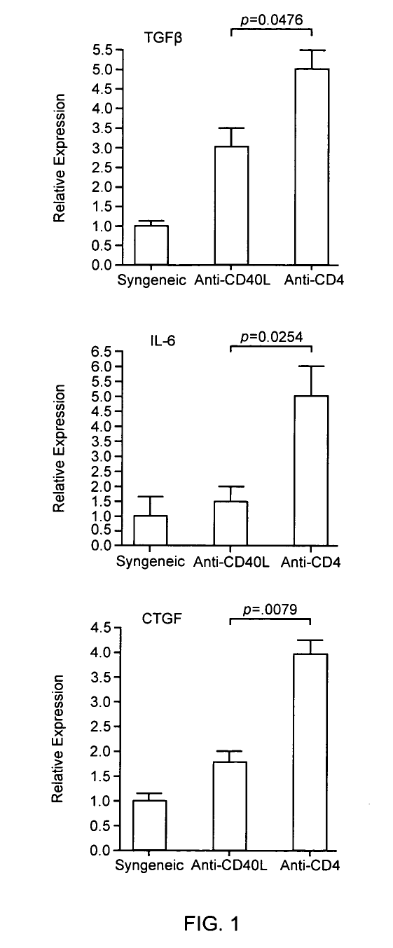

Transduction of allografts, but not syngeneic grafts, with TGFβ is sufficient to induce CTGF and CR (Csencsits, et al. 2006) indicating the involvement of an immune component in TGFβ-mediated fibrosis. TGFβ, CTGF, and IL-6 (a cytokine recently identified to have a role in CR) transcripts were measured by quantitative real time PCR, as described in Methods and Materials above, the mouse model described in example 1. Expression levels in allografts whose recipients were transiently depleted of CD4+ cells, which develop CR, were compared to levels in allografts whose recipients were treated with anti-CD40L, which do not develop CR, or to levels in untreated syngeneic grafts. Intragraft levels of TGFβ, IL-6, and CTGF were significantly increased (p=0.0476, p=0.0254, and p=0.0079 respectively) in cardiac allografts whose recipients were transiently depleted of CD4+ cells compared to grafts whose recipients were treated with anti-CD40L or to syngeneic controls (FIG. 1). Thus, the upregulation of all three cytokines was observed in cardiac allograft grafts undergoing CR, but not in syngeneic cardiac grafts (which do not exhibit CR) or in cardiac allografts not undergoing CR. This experiment indicated that the increased expression of CTGF correlates with the appearance of chronic rejection in cardiac allografts.

### Example 3

### Forced Expression of CTGF or TGF3 Promotes Allograft Fibrosis

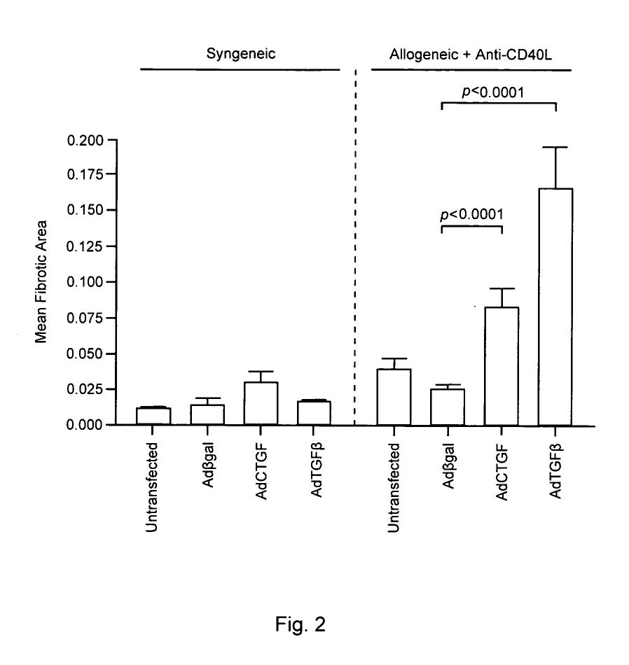

To determine whether exogenous expression of CTGF promotes cardiac fibrosis, allografts and syngeneic grafts were transduced with AdCTGF as described in Methods and Materials, above. AdCTGF transduction of allografts in recipients treated with anti-CD40L (which do not undergo CR in the mouse model) caused a significant increase in fibrotic area by day 30 post transplant compared to allografts transduced with control AdβGal virus (FIG. 2). In contrast, syngeneic grafts transduced with AdCTGF had similar levels of fibrosis to the untransduced and AdβGal controls. It should be noted that the mean fibrotic area for AdCTGF-transduced allografts was less than in hearts transduced with AdTGFβ, consistent with previous descriptions in lung transductions (Bonniaud P et al Am J Respir Crit Care Med 2003 168(7):770-778). This difference could not be accounted for by differences in transgene expression levels, as AdTGFβ and AdCTGF expression were comparable in these studies as determined by real time PCR (data not shown). Thus, while forced expression of either TGFβ or CTGF promoted cardiac allograft fibrosis, they did so to different extents (FIG. 2). This could in part be due to TGFβ induction of endogenous CTGF expression (Csencsits K et al. Am J Transplant 2006 6(5 Pt 1):959-966; Chen M M et al. J Mol Cell Cardiol 2000 32(10):1805-1819; Grotendorst G R et al. Cell Growth Differ 1996 7(4):469-480), thereby producing an additive effect.

It has been observed previously that, in some settings, TGFβ and CTGF are potently fibrotic in tandem while less fibrotic alone (Frazier K et al. J Invest Dermatol 1996 107(3):404-411; Mori T et al. J Cell Physiol 1999 181(1):153-159). However, no increases in fibrosis were observed upon co-transduction of syngeneic grafts compared to single virus transduction (data not shown). Thus, while injection of TGFβ and CTGF synergize to cause fibrotic responses in the skin (Mori T et al. J Cell Physiol 1999 181(1):153-159), forced expression of both was insufficient to induce fibrosis or CR in syngeneic cardiac grafts, further supporting the requirement of an immune component.

### Example 4

### CTGF Neutralization Ameliorates Allograft Fibrosis

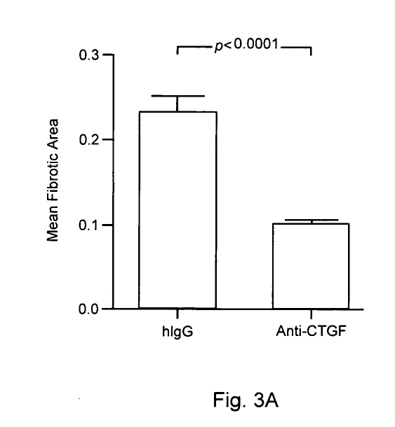

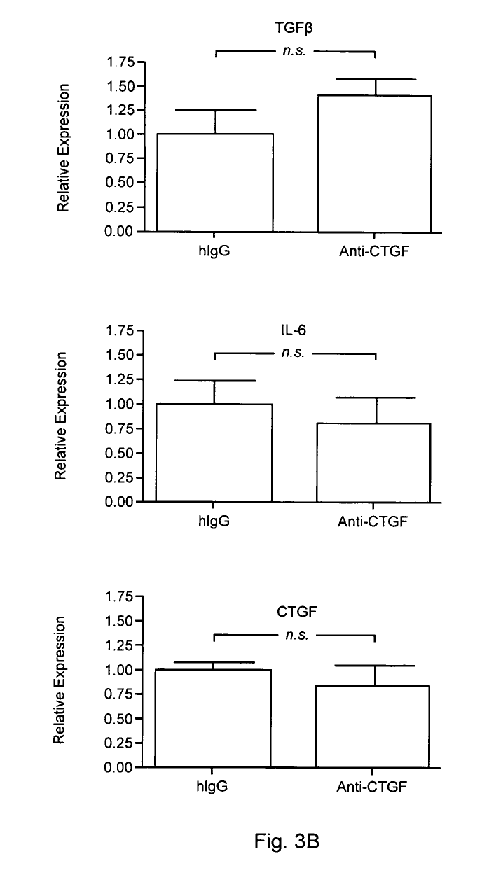

To determine whether CTGF neutralization would inhibit the fibrosis associated with CR, cardiac allograft recipients that were transiently depleted of CD4+ cells, using anti-CD4 antibody as described in Example 1, were treated with neutralizing anti-CTGF mAb (CLN1) or control antibody (human IgG). Treatment with anti-CTGF mAb resulted in significant reduction of fibrotic area compared to control antibody treatment (p<0.0001, FIG. 3A). The reduction in fibrosis associated with anti-CTGF treatment was not accompanied by reduction of intragraft TGFβ, CTGF, or IL-6 transcripts (FIG. 3B). This experiment shows that treatment with a neutralizing anti-CTGF antibody reduces the fibrosis associated with chronic allograft rejection.

### Example 5

### CTGF Neutralization Decreases Cardiomyocyte Hypertrophy Associated with CR

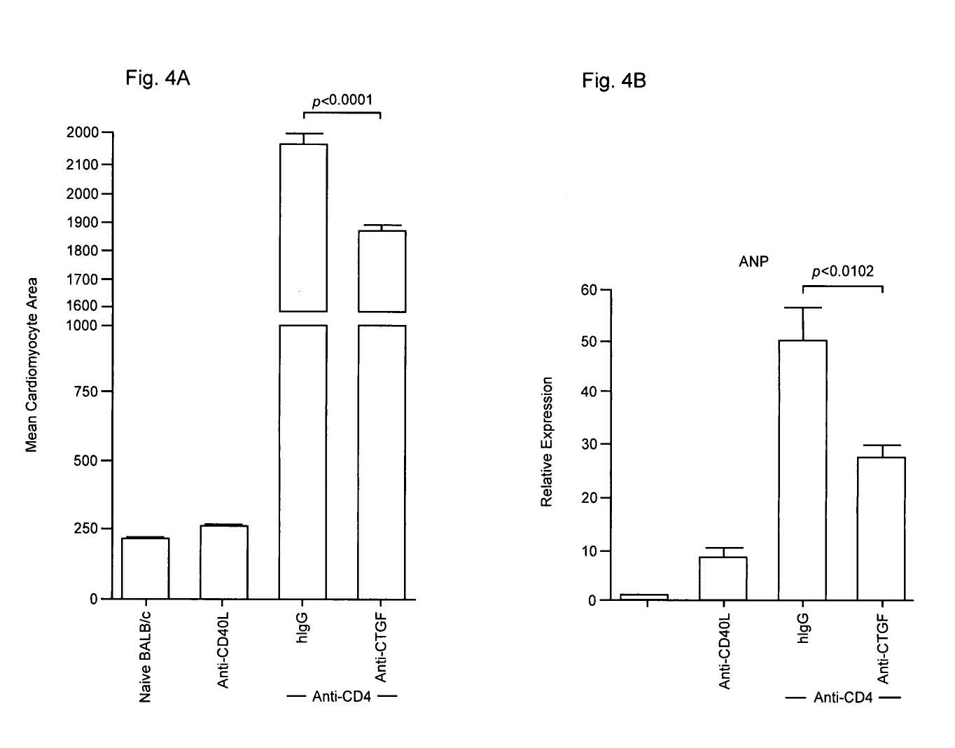

In addition to promoting fibrosis, CTGF can induce cardiomyocyte hypertrophy (Hayata N, Fujio et al. Biochem Biophys Res Commun 2008 370(2):274-278; Matsui Y and Sadoshima J. J Mol Cell Cardiol 2004 37(2):477-481), a function it shares with IL-6. The effect of neutralizing CTGF activity with the anti-CTGF mAb on cardiomyocyte hypertrophy was assessed. Cardiac allografts whose recipients were treated with anti-CTGF mAb (CLN1) as described in Example 4 exhibited a significant decrease (p<0.0001) in cardiomyocyte hypertrophy as determined by cross-sectional measurement of individual cardiomyocytes on H&E stained sections (FIG. 4A). Further, anti-CTGF mAb treatment significantly reduced (p=0.0102) intragraft atrial natriuretic peptide (ANP) expression (FIG. 4B), a molecular marker of cardiac hypertrophy (Caron K M et al. Proc Natl Acad Sci USA 2004 101(9):3106-3111; Fredj S et al. J Cell Physiol 2005 202(3):891-899). ANP expression was measured by quantitative real time PCR as described in Methods and Materials above. These findings support a role for anti-CTGF agents in ameliorating not only fibrosis, but also pathologic hypertrophy, associated with CR. For reference, cardiomyocyte area and ANP expression levels for naïve, untransplanted BALB/c hearts and cardiac allografts transplanted into recipients receiving anti-CD40L therapy are shown.

### Example 6

### CTGF Neutralization Inhibits T Cell Infiltration of Grafts

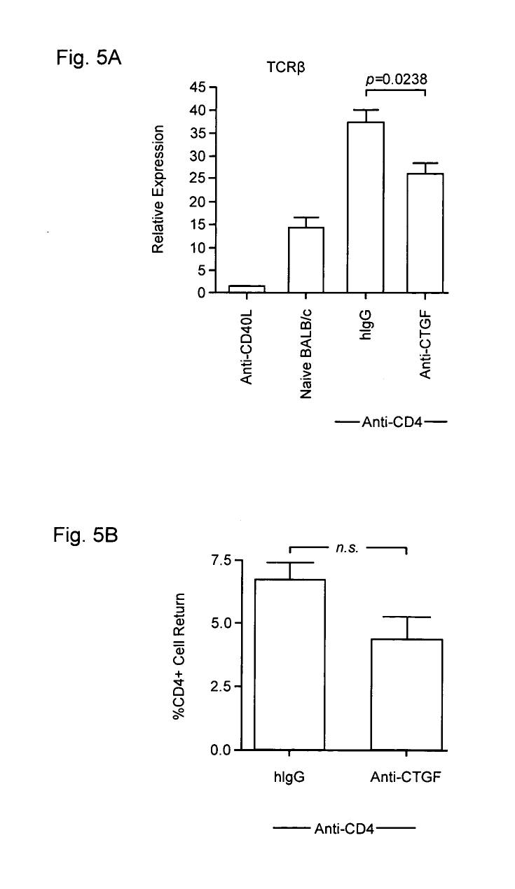

Multiple studies have suggested an essential role for CTGF in promoting integrin-mediated adhesive responses in multiple cell types (Babic A M et al. Mol Cell Biol 1999 19(4):2958-2966; Ball D K et al. J Endocrinol 2003 176(2):R1-7; Chen C C et al. J Biol Chem 2001 276(13):10443-10452; Chen Y et al. Mol Biol Cell 2004 15(12):5635-5646; Gao R and Brigstock D R. J Biol Chem 2004 279(10):8848-8855; Hoshijima M et al. FEBS Lett 2006 580(5):1376-1382; Nishida T et al. J Cell Commun Signal 2007 1(1):45-58; Gao R, and Brigstock DR. Gut 2006 55(6):856-862; Heng E C et al. J Cell Biochem 2006 98(2):409-420; Jedsadayanmata A et al. J Biol Chem 1999 274(34):24321-24327). Further, CTGF has been demonstrated to induce the production of chemokines that can attract monocytes, macrophages, T cells, and natural killer cells (Wu S H et al. Growth Factors 2008:1). The effect of CTGF neutralization on the infiltration of immune cells into grafts undergoing CR was evaluated. Representative H&E stains of day 30 post transplant cardiac allografts taken from recipients transiently depleted of CD4+ cells (Anti-CD4) and receiving CTGF neutralizing mAb (Anti-CTGF) or control antibodies (hIgG) suggest a reduction in perivascular infiltrate density in grafts treated with neutralizing Anti-CTGF antibody (CLN1). Histologic analysis suggested that less cellular infiltrate was present in grafts receiving anti-CTGF antibody compared to control antibody. Consistent with histologic observations, a significant decrease (p=0.0238) in TCRβ constant region expression, a marker of graft infiltrating T cells (Zhao X M et al. Clin Exp Immunol 1993 93(3):448-451), was observed (FIG. 5A). To verify that this difference was not due to CTGF neutralization preventing peripheral repopulation of CD4+ cells, CD4+ cells in anti-CTGF and control treated graft recipients were compared. No significant differences were observed between these groups (FIG. 5B). This experiment shows that treatment with the anti-CTGF antibody reduces the perivascular infiltrate density in cardiac allografts.

Various modifications of the invention, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method for treating or preventing chronic allograft rejection in a subject having received or about to receive an allograft, wherein the method comprises administering a therapeutically effective amount of an anti-CTGF antibody to said subject, wherein said antibody is CLN1.

2. A method for prolonging the survival of an allograft in an allograft recipient, wherein the method comprises administering a therapeutically effective amount of an anti-CTGF antibody to said recipient, wherein said antibody is CLN1.

3. A method of reducing or preventing fibrosis in an allograft, wherein the method comprises administering a therapeutically effective amount of an anti-CTGF antibody to said allograft, wherein said antibody is CLN1.

4. A method of reducing or preventing transplant-associated hypertrophy in an allograft, wherein the method comprises administering a therapeutically effective amount of an anti-CTGF antibody to said allograft, wherein said antibody is CLN1.

5. The method of any one of claims 1-4, wherein said allograft comprises transplanted tissue selected from the group consisting of heart, lung, liver, kidney, intestine, eye, cornea, skin, ligament, tendon, bone, and pancreas.

6. The method of claim 5, wherein the allograft is a cardiac allograft.

7. The method of any one of claims 1-4, wherein the anti-CTGF antibody is administered beginning in the time period between 7 and 21 days post-transplantation.

8. The method of any one of claims 1-4, wherein the anti-CTGF antibody is administered in combination with one or more therapeutics selected from the group consisting of an immune suppressant, corticosteroid, calcineurin inhibitor and antimetabolite.

9. The method of claim 1, wherein the anti-CTGF antibody is administered before chronic rejection is detected.

10. The method of claim 1, further comprising the step of determining the extent of the allograft rejection prior to administering the anti-CTGF antibody.

11. The method of claim 1, further comprising the step of determining the extent of the allograft rejection subsequent to administering the anti-CTGF antibody.

12. The method of claim 2, further comprising the step of comparing the post-transplant survival rate of said allograft to a standard post-transplant survival rate.

13. The method of claim 3, further comprising the step of determining the amount of fibrosis in said allograft prior to administering the anti-CTGF antibody.

14. The method of claim 3, further comprising the step of determining the amount of fibrosis in said allograft subsequent to administering the anti-CTGF antibody.

15. The method of claim 4, further comprising the step of determining the amount of hypertrophy in said allograft prior to administering the anti-CTGF antibody.

16. The method of claim 4, further comprising the step of determining the amount of hypertrophy in said allograft subsequent to administering the anti-CTGF antibody.


SEQUENCE LISTING (14 sequences)

SEQ ID NO: 1 - DNA, 19 bp, Artificial Sequence
  Synthetic
ggaggtcaacccacctctg

SEQ ID NO: 2 - DNA, 22 bp, Artificial Sequence
  Synthetic
gctccaatcctgtcaatcctac

SEQ ID NO: 3 - DNA, 23 bp, Artificial Sequence
  Synthetic
ggaaaacattaagaagggcaaaa

SEQ ID NO: 4 - DNA, 20 bp, Artificial Sequence
  Synthetic
ccgcagaacttagccctgta

SEQ ID NO: 5 - DNA, 20 bp, Artificial Sequence
  Synthetic
ctggtgctgagtatgtcgtg

SEQ ID NO: 6 - DNA, 20 bp, Artificial Sequence
  Synthetic
cagtcttctgagtggcagtg

SEQ ID NO: 7 - DNA, 23 bp, Artificial Sequence
  Synthetic
cgtggaaatgagaaaagagttgt

SEQ ID NO: 8 - DNA, 21 bp, Artificial Sequence
  Synthetic
tccagtttggtagcatccatc

SEQ ID NO: 9 - DNA, 21 bp, Artificial Sequence
  Synthetic
cctgagtggctgtcttttgac

SEQ ID NO: 10 - DNA, 21 bp, Artificial Sequence
  Synthetic
cctgtattccgtctccttggt

SEQ ID NO: 11 - DNA, 19 bp, Artificial Sequence
  Synthetic
ggactctccaccgcaatga

SEQ ID NO: 12 - DNA, 20 bp, Artificial Sequence
  Synthetic
gaccaggatctcttgctgga

SEQ ID NO: 13 - DNA, 19 bp, Artificial Sequence
  Synthetic
ctgccaagtgcagttccat

SEQ ID NO: 14 - DNA, 20 bp, Artificial Sequence
  Synthetic
ggcctctgcactgatgttct